(19) United States
(12) Patent Application Publication
(10) Pub. No.: US 2011/0009374 A1
(43) Pub. Date: Jan. 13, 2011

(54) METHOD OF WOUND HEALING AND SCAR MODULATION

(76) Inventor: Brian C. KELLER, Antioch, CA (US)

Correspondence Address: MORRISON & FOERSTER LLP, 12531 HIGH BLUFF DRIVE, SUITE 100, SAN DIEGO, CA, 92130-2040, US

(21) Appl. No.: 12/833,902

(22) Filed: Jul. 9, 2010

Related U.S. Application Data
(60) Provisional application No. 61/224,408, filed on Jul. 9, 2009.
(60) Provisional application No. 61/226,216, filed on Jul. 16, 2009.

(51) Int. Cl. A61K 31/573; A61P 17/02

(52) U.S. Cl. 514/179; 514/177

(57) ABSTRACT

The invention relates to methods of promoting wound healing and reducing scar formation by administration of corticosteroids, and pharmaceutical compositions comprising corticosteroids.

### RELATED APPLICATIONS

This application claims priority to U.S. Provisional Nos. 61/224,408, filed Jul. 9, 2009, and 61/226,216, filed Jul. 16, 2009. Both applications are hereby incorporated by reference in their entirety.

### FIELD OF THE INVENTION

The present invention relates to pharmaceutical compositions containing corticosteroids and methods of using such compositions to promote wound healing and reduce scar formation. In particular, the invention relates to use of corticosteroids formulated with silicone crosspolymers for such purposes.

### BACKGROUND

Scarring results from a normal physiological healing response after skin injury or incision. The skin wound healing process consists of three phases—inflammation, granulation and matrix remodeling. During the first phase, an inflammatory response is mounted, producing a cascade of biochemical reactions that result in vasodilation, exudate filling of the wound, and swelling at the site of injury. Neutrophil migration into the area of injury triggers phospholipase A₂ (PLA₂) release and prostaglandin production which lead to cellular and tissue damage. In the second phase, granulation takes place as macrophages secrete cytokines to promote granulated tissue formation. This new tissue consists of new epithelial tissue complete with new vasculature and blood supply. In phase three, matrix remodeling occurs as fibroblasts proliferate and manufacture collagen, elastin and other tissue building blocks in and around the wound site.

The end product of wound healing is neither aesthetically nor functionally perfect. Unwounded dermis comprises a mechanically efficient basket-weave meshwork of collagen. However, wound healing in mammalian skin results in varying degrees of scar formation, ranging clinically from fine asymptomatic scars to problematic hypertrophic and keloid scars, which may limit function, restrict further growth, or have a poor cosmetic appearance. A healed wound retains a connective tissue scar where the collagen matrix has been poorly reconstituted in dense parallel bundles. While cells of the dermis and epidermis will repopulate after wounding, epidermal appendages lost at the site of damage do not regenerate. The resulting tissue comprises high amounts of densely layered collagen assembled in no apparent architectural scheme. A major goal of wound-healing biology is to determine how skin can be induced to reconstruct the damaged parts more perfectly. Brown, B. C. et. al. “The hidden cost of skin scars: quality of life after skin scarring,” J. Plast. Reconstr. Aesthet. Surg. 2008, 61, 1049-1058; Martin, P. et al. “Wound healing—aiming for perfect skin,” Science 1997, 276(5309), 75-81.

Hypertrophic scars represent a frequent but exaggerated response to healing. See, generally, Lewis, W. H. and K. K. Sun. “Hypertrophic scar: a genetic hypothesis.” Burns 1990, 16(3), 176-78; Xie, J. et al. “Effects of antisense oligodeoxynucleotide to type I collagen gene on hypertrophic scars in the transplanted nude mouse model,” J. Cutan. Pathol. 2009, 36(11), 1146-50. Clinically, hypertrophic scars are raised, red and often nodular. They occur in all skin areas but are most common in areas of thick skin. Frequently, hypertrophic scars develop within weeks of a burn, wound closure, wound infection, hypoxia or other traumatic skin injury. Collagen found in this type of scars in highly disorganized and forms whorl like arrangements rather than normal parallel patterns, causing induration and elevation above the normal skin surface.

Current treatment options range from no treatment at all (i.e., leaving the scar alone), to invasive procedures and surgery such as intralesional corticosteroids, laser therapy and cryosurgery, to noninvasive management, particularly through topical medications. See, e.g., Zurada et al., J. Am. Acad. Dermatol. 55:1024-31 (2006); Meier, K. and L. B. Nanney. “Emerging new drugs for wound repair.” Expert Opin. Emerg. Drugs 2006, 11(1), 23-37. Most scar sufferers who elect to undergo treatment prefer noninvasive techniques; overall compliance with these techniques is higher because the therapy is self-controlled and less painful. Epidermal wound healing may also be affected by topically applied agents which change the physical environment. For example, it has been demonstrated that simple occlusion with polyethylene film or silicone film increased the rate of histologically confirmed epidermal wound healing in animals and man.

One of the important materials used in noninvasive management of scars has been polydimethylsiloxane polymer gel sheeting or silicone gel sheeting. Quinn, K. J. “Silicone gel in scar treatment.” Burns Inc. Therm. Inj. 1987, 13 Suppl., S33-40. Since being introduced in 1982, topical silicone gel sheeting has been used to minimize the size, induration, erythema, pruritus, and extensibility of pre-existing hypertrophic scars, with mixed results. See, e.g., Fette, Plastic Surg. Nurs. 26:87-92 (2006); de Oliveria et al., Dermatol. Surg. 27:721-26 (2001); Ricketts et al., Dermatol. Surg. 22:955-59 (1996). However, controlled studies have demonstrated no significant differences between gel wound dressing and silicone-based wound dressings. Moreover, use of silicone gel sheeting is problematic and thus, suffers from a high non-compliance rate. By their nature silicone gels are difficult to handle. They are soft and frangible and the gel sheets are thus easily torn in use. The strength and ease of handling of silicone gel sheets may be improved by embedding therein during manufacture a support material such as a net of polyester or other fibers. This technique has resulted in an improvement in the ability to handle and apply the gel sheet, but the sheet still has a tendency to fragment during application and use. The sheeting also must be worn up to 24 hours a day for 2-4 months.

Seeking to avoid some of the constraints faced by silicone gel sheeting, liquid silicone gel products have also been tried. Liquid dimethicone products, for example, are easy to use but again, compliance is low due to the unappealing greasy, messy nature of liquid dimethicone. Attempts to reduce or eliminate the messy nature of silicone largely depend on complicated wound dressing formulations that lack the necessary conformability and long-term flexibility necessary for most wounds.

Epidermal healing has traditionally been viewed in terms of different phases, including proliferation, migration, matrix synthesis, and contraction. Studies of the fast and efficient dermal regeneration processes in embryos have begun to indicate ways the normal adult repair process might be adjusted to mimic regeneration. See, e.g., Adzick N. S. and H. P. Lorenz. “Cell, matrix, growth factors, and the surgeon: the biology of scarless fetal wound repair,” Ann. Surg. 1994, 220(1), 10-18. New efforts look beyond the discrete healing phases to focus on specific biochemical mechanisms of wound healing, which are complex cell signal-mediated processes that rely on the collaboration of many different tissues, growth factors, and cell lineages at different points during the healing phases. Skin wound healing in adult mammals is a complex process requiring the collaborative efforts of many different tissues and cell lineages. The behavior of the various cell types during the healing phases, including the functions of growth factor and matrix signals at a wound site, are only roughly understood. A number of dermal cell types and growth factors have been identified, including fibroblasts, keratinocytes, endothelial cells, inflammatory cells, epidermal growth factor (EGF), Transforming Growth Factor (TGF-α), and Heparin Binding EGF (HB-EGF), all of which are known to assist in the regeneration process.

It is well known that inflammation-mediated release of biochemical modulators plays a significant role in the rate and quality of wound healing both locally and systemically. For example, if inflammation spreads systemically as a result of bacterial infection in the wound, the patient is at risk for physiologic and metabolic changes, including sepsis, which can cause multisystem organ failure and death. See, Annane, D. “Sepsis clinical knowledge: a role of steroid treatment,” Minerva Anestesiol. 2003, 69(4), 254-7.

The present invention involves a method of enhancing the scar healing process with topically applied corticosteroids. The utility of corticosteroids may stem from their diverse functions and tissue distribution. Corticosteroids modulate carbohydrate, protein and lipid metabolism, and help preserve normal function of multiple organ systems, such as the cardiovascular system, the immune system, the kidneys, the skeletal muscle, the endocrine system and the nervous system. Although the mechanisms of corticosteroid activity are not well understood, these compounds are known primarily as gene regulators. Inside cells, corticosteroids' primary action involves interaction with specific receptor proteins in target tissues to regulate the expression of corticosteroid-responsive genes, and thus, the levels and array of proteins synthesized by the target tissues. Corticosteroids generally increase the expression of target genes, although there are well-documented examples in which such compounds decrease transcription.

Through cell membrane interactions, corticosteroids also mediate anti-inflammatory effects; for example, corticosteroids prevent phospholipid conversion and cause a decrease in eosinophil action. In a particular biochemical example, glucocorticoids control anti-inflammatory responses through the lipocortin-1 (annexin-1) synthesis pathway. Lipocortin-1 both suppresses phospholipase A₂, thereby blocking eicosanoid production and inhibiting various leukocyte inflammatory events. Thus, glucocorticoids modulate the immune response by inhibiting the production two main products of inflammation, prostaglandins and leukotrienes. See, generally, Francois B. et. al. “12-h treatment with methylprednisolone versus placebo for prevention of postextubation laryngeal oedema: a randomized double-blind trial,” Lancet, 2007, 369(9567), 1083-89.

Systemic administration of glucorticosteroids downregulates the immune response, leading to a decrease in the number of circulating lymphocytes, eosinophils, monocytes and basophils. At the same time, corticosteroids increase polymorphonuclear leukocytes and increase the demargination of vascular walls. Certain malignancies, such as lymphoid tumors, are destroyed by corticosteroid treatment. As a result, there is decreased release of vasoactive and chemoattractive factors, diminished secretion of lipolytic and proteolytic enzymes, decreased extravasation, decreased migration of leukocytes to areas of injury, and, ultimately, decreased fibrosis and fibrotic characteristics to the ensuing scar tissue. Because of these immune system depressant effects, and the known importance of inflammatory mediators to local and systemic healing processes, corticosteroids have been viewed as having a negative effect on wound healing, particularly with respect to healing time.

Nevertheless, intralesional injection of corticosteroids has been studied extensively in scar treatment. Jalali, M. and A. Bayat. “Current use of steroids in management of abnormal raised skin scars,” Surgeon 2007, 5(3), 175-80; Khoo, C. “A simple new technique for injecting steroids into scars,” Ann. Plast. Surg. 1987, 19(3), 291-3. After an intralesional injection, a decrease in erythema, swelling, heat, and tenderness may be observed. Studies show immediate post-operative corticosteroid injection may prevent keloid scar formation. Jung, J. Y. et al., Ann. Dermatol. 2009, 21(3), 221-225. However, such procedures suffer from low response rates, risk of infections, lack of patient compliance, the need for the medications to be administered in a physician's office, pain associated with the injections, and increased medical costs. Although topical application of corticosteroids would avoid such negative consequences, the topical use of such compounds on fresh wounds has not been generally advocated as a wound treatment. In fact, the literature is conflicting on this issue and several studies have shown topical treatment with corticosteroids to be ineffective or even contraindicated. See, e.g., Jenkins, M. et al. Failure of topical steroids and vitamin E to reduce postoperative scar formation following reconstructive surgery. J. Burn Care Rehabil. 1986, 7(4), 309-312; Mustoe, T. Scars and keloids. Br. Med. J. 2004, 328, 1329-30. For example, Riaz and colleagues determined topical corticosteroids have no effect on procollagen activity. Riaz, Y. et al. Type 1 procollagen as a marker of severity of scarring after sternotomy: effects of topical corticosteroids. J. Clin. Pathol. 1994, 47, 892-899. Even where topical corticosteroids were found to inhibit certain inflammatory processes such as fibroblast growth, there is no indication the steroids had any effect on scar healing time. See Berkliner, D. L. et al. Decreased scar formation with topical corticosteroid treatment. Surgery 1967, 61(4), 619-625. Thus, the link between the biochemical effects of corticosteroids on inflammatory processes and any medically or cosmetically significant aspect of scar healing has remained elusive. For additional discussion of topical treatments, see Baumann, L. S. et al. “The effects of topical vitamin E on the cosmetic appearance of scars.” Dermatol. Surg. 1999, 25(4), 311-5; Waymack, P. J. et al. “Evaluation of the effect of topical steroids on human scar contracture using a nude mouse model,” J. Burn Care Rehabil. 1988, 9(6), 640-42.

Therefore, there remains a need for improved topical wound or scar treatments with improved scar healing properties.

### SUMMARY OF THE INVENTION

The present invention has found, contrary to the teachings described above, that application of a low-potency corticosteroid, e.g., methylprednisolone acetate or prednisolone acetate, to wounded epidermis actually causes faster healing with less scarring when compared to an untreated wound. The topical application of low doses of corticosteroids to a wound appears to mediate and regulate the healing process to the point where more normal epidermal tissue is laid down and less scarring occurs. While topical corticosteroids have been thought to slow the rate of wound healing in animal and human skin, as discussed above, such treatment may actually produce better wound margin repair and controlled immunoregulation supporting the regeneration of a normal epidermal layer. Remedies that affect these biochemical processes may in fact allow more efficient control over the healing process, resulting in recovered dermal layers with more natural functional and aesthetic characteristics. The present invention demonstrates topical corticosteroids have a positive effect on the healing process.

Furthermore, it has been surprisingly discovered that a topical formulation comprising a corticosteroid and a high molecular weight, low viscosity silicone crosspolymer provides optimal therapeutic benefits in terms of scar healing time, redness, topography, erythma, and other parameters. Such formulations are particularly useful when they further comprise an additional anti-inflammatory agent. Once the formulation is applied to the skin, the silicone crosspolymers cure rapidly at room temperature to provide a conformable, highly flexible, medicated dressing that can cover the closed wound or scar for extended periods of time. The formulations are easily applied to closed wounds without causing additional injury to the affected area; they are soothing to the scar tissue, painless, and free from side effects. Patient compliance is high because the formulations are not greasy, go on dry, and occlude the scar. The formulations provided herein minimize further scarring, reduce potential infections, minimize induration and hypertrophy, and diminish scar discoloration. The corticosteroid-silicone crosspolymer formulations described herein are particularly useful because the silicone mixture dissolves the corticosteroid to allow for better drug activity in the skin. The use of such silicone crosspolymers in scar treatment has been described, see WO 2008/109887 and U.S. patent application Ser. No. 12/555,749.

In comparison to intralesional corticosteroid injections, the present topical formulation can reduce the risk of wound infection, is soothing to the wound, helps protect the wound from air (which causes irritation, drying, flaking, and discomfort), can be applied by the patient without medical intervention, and provides a significantly more cost effective wound treatment device. The topical formulation is better suited for pediatric use, and avoids the psychological aversion many patients have to receiving an injection into to a fresh wound.

Thus, in one aspect, the invention is directed to a method of treating a subject with a wound or a scar comprising topically administering to the wound or scar an effective amount of a pharmaceutical composition comprising a corticosteroid.

In a second aspect, the invention is directed to a pharmaceutical composition comprising a corticosteroid and a high molecule weight, low viscosity silicone crosspolymer.

### DETAILED DESCRIPTION OF THE INVENTION

For the sake of brevity, the disclosures of publications, including patents and published patent applications, cited in this specification are incorporated by reference herein. Unless defined otherwise, all technical and scientific terms used herein have the same meaning as is commonly understood by one of ordinary skill in the art to which this invention belongs. If a definition set forth in this section is contrary to or otherwise inconsistent with a definition set forth in the patents, applications, published applications and other publications that are incorporated by reference herein, the definition set forth in this section prevails over the definition that is incorporated by reference.

As used herein, the terms “including,” “containing,” and “comprising” are used in their open, non-limiting sense.

As used herein, “a” or “an” means “at least one” or “one or more.”

For any quantitative expression used herein, it is understood the quantity is meant to refer to the actual value and is also meant to refer to the approximation of the value that would be inferred by one of skill in the art, including approximations due to the experimental and/or measurement conditions for the given value. This inference is intended regardless of whether the term “about” is used explicitly with the quantity or not.

The term “corticosteroid,” as used herein, refers to natural steroid hormones or synthetic variants thereof. The term is intended to include corticosteroids, glucocorticosteroids (or glucocorticoids), and mineralocorticoids. Such compounds are known to control carbohydrate, fat, and protein metabolism and to mediate inflammatory responses. Particularly preferred are corticosteroids which are soluble in the chosen topical formulation. The term “corticosteroid” also includes a pharmaceutically acceptable salt form of any such compound or derivative. A “pharmaceutically acceptable salt” is intended to mean a salt of a free acid or base of a compound represented herein that is non-toxic, biologically tolerable, or otherwise biologically suitable for administration to the subject. See, generally, S. M. Berge, et al., “Pharmaceutical Salts,” J. Pharm. Sci., 1977, 66, 1-19. Preferred pharmaceutically acceptable salts are those that are pharmacologically effective and suitable for contact with the tissues of subjects without undue toxicity, irritation, or allergic response.

Examples of suitable corticosteroids include methylprednisolone, prednisolone, hydrocortisone, cortisone, tixocortol, prednisone, mometasone, amcinonide, budesonide, desonide, betamethasone, dexamethasone, prednicarbate, fluocortolone, clobetasone, clobetasol, fluprednidene, clobetasol, halobetasol, diflorasone, fluocinonide, halcinonide, triamcinolone, desoximetasone, fluocinolone, flurandrenolide, fludrocortisone, fluticasone, desonide, prednicarbate, difluocortolone, and derivatives thereof. Suitable derivatives include acetates, propionates, butyrates, caproates, valerates, pivalates, acetonides, aceponates, buteprates, furoates, and combinations thereof. In some embodiments, selected corticosteroids and derivatives thereof include aclometasone dipropionate, amcinonide, betamethasone dipropionate, betamethasone sodium phosphate, betamethasone valerate, budesonide, clobetasol-17-propionate, clobetasone-17-butyrate, cortisone acetate, desonide, dexamethasone, dexamethasone sodium phosphate, fluocinolone acetonide, fluocinonide, fluocortolone, fluocortolone caproate, fluocortolone pivalate, fluprednidene acetate, halcinonide, hydrocortisone, hydrocortisone acetate, hydrocortisone-17-butyrate, hydrocortisone-17-valerate, methylprednisolone, methylprednisolone acetate, mometasone, prednisolone, prednisolone acetate, prednisone, tixocortol pivalate, triamcinolone acetonide, and triamcinolone alcohol. In particular embodiments, the corticosteroid is methylprednisolone acetate, prednisolone acetate, or triamcinolone acetonide. In other embodiments, the corticosteroid is prednisolone acetate or methylprednisolone acetate. In further embodiments, the corticosteroid is prednisolone acetate. In still other embodiments, the corticosteroid is methylprednisolone acetate.

As used herein, the term “wound” refers to an injury to the dermis of the skin of a subject in which skin is torn, cut, or punctured. Wounds typically include open wounds such as incisions, cuts, lacerations, abrasions, puncture wounds, traumatic skin injury, penetration wounds, burns, and the like. Wounds may be chronic, e.g., resulting from disease or other slow tissue damage, or acute, e.g., resulting from an accident, injury, or surgical procedure. Wounds may be caused by lasers during, for example, medical procedures, dermatological surgery, or cosmetic surgery. Wounds may also result from the inflammatory and pruritic manifestations of corticosteroid-responsive dermatoses (for example, atopic dermatitis or other dermatoses), including those associated with the formation of scar tissue.

The term “scar,” as used herein, refers to dermal tissue that results from wound healing, typically comprising fibrous tissue. A scar may result from any of the etiologies described for the term “wound” and thus, these two terms are generally used interchangeably herein. The term “scar” covers hypertrophic scars, keloid scars, contracture scars, and other types of scars such as atrophic scars. Symptoms of scars include skin discolorations (including redness, changes in pigmentation, or other discolorations), erythma, dry, flaky, or itchy skin, raised area above the surrounding skin, keloid formation, hypertrophy, scar pain, decreased vascularity of the scar and/or surrounding tissue, reduced pliability, and poor aesthetic appearance (including quality and texture of the scar tissue). Scars resulting from any type of wound may be treated in accordance with the present invention. The corticosteroids and formulations described herein are particularly suitable for treatment of hypertrophic scars resulting from burn injuries.

The pharmaceutical compositions described herein may be formulated as solutions, emulsions, suspensions, or dispersions in suitable pharmaceutical bases or carriers, according to conventional methods known in the art for preparation of various dosage forms. For the topical applications described herein, corticosteroids may be formulated as gels, creams, pastes, lotions, or ointments or as a similar vehicle suitable for topical administration. Topical administration may also be effected through the use of liposomal or dermal patch delivery systems. Corticosteroids may be formulated for transdermal or interdermal delivery or in an extended release formulation. For example, suitable corticosteroid formulations may employ liposomes or similar lipid-based vesicles to enhance stability of the product or to provide for extended release of the drug to the affected area. Any suitable liposome or liposome composition may be employed. Exemplary liposomes include those described in U.S. Pat. Nos. 6,958,160 and 7,150,883, and may comprise one or more fatty acid-diacylglycerol-PEG derivatives such as PEG-12 glyceryl dioleate, PEG-23 glyceryl distearate, PEG-12 glyceryl dipalmitate, or PEG-12 glyceryl dimyristate. Other examples of suitable liposomes are those made from conventional phospholipids derived from egg lecithin or soy lecithin.

Thus, for treatment purposes, a pharmaceutical composition (or “formulation”) comprising a corticosteroid may further comprise one or more pharmaceutically acceptable excipients. A pharmaceutically acceptable excipient is a substance that is non-toxic and otherwise biologically suitable for administration to a subject. Such excipients facilitate administration of and are compatible with the corticosteroid. Examples of pharmaceutically acceptable excipients include stabilizers, thickeners, lubricants, surfactants, diluents, anti-oxidants, binders, preservatives, coloring agents (such as pigments or dyes), or emulsifiers. Pharmaceutical excipients may also include skin permeation enhancers. Stabilizers specifically include amine stabilizers. Suitable thickeners are the swelling agents customarily used for gel formation in galenic pharmacy. Examples of suitable thickeners include natural organic thickeners, such as agar-agar, gelatin, gum arabic, a pectin, and the like, modified organic natural compounds, such as carboxymethylcellulose or cellulose ethers, or fully synthetic organic thickeners, such as polyacrylic compounds, vinyl polymers, or polyethers. In some embodiments, the excipient can increase the smoothness or other properties of the scar dressing formulation. Such additives include, but are not limited to glycerin, propylene glycol, butylene glycol, esters, diacyl glycerol esters, and starch. In certain embodiments, pharmaceutical compositions are sterile compositions.

In particular embodiments, the pharmaceutically acceptable excipient is purified water, ethanol, ethoxydiglycol, butylene glycol, carbopol ETD 2001, citric acid, isopropyl palmitate, caprilic/capric triglyceride, sorbitan stearate, corn oil, stearic acid, cetyl alcohol, glyceryl stearate, PEG-100 stearate, methylparaben, propylparaben, oleic acid, phenoxyethanol, carbopol Ultrez 10, glycerin, carbopol ETD 2020, propylene glycol, cholesterol, trolamine, ammonium acryloyldimethyltaurate/VP copolymer, or benzyl alcohol, or a mixture thereof.

In particular embodiments, corticosteroid formulations further comprise silicone-derived materials such as silicone crosspolymers and silicone oils. Silicones are a group of completely synthetic polymers containing the recurring group —SiR₂O—, wherein R is a radical such as an alkyl, aryl, phenyl or vinyl group. The simpler silicones are oils of very low melting point, while at the other end of the scale of physical properties are highly crosslinked silicones which form rigid solids. Intermediate in physical properties between these two extremes are silicone crosspolymers which are gels and rubbers. Silicone crosspolymers are formed by crosslinking a mixture of two or more silicones; the various molecular weights of the individual components and/or their degree of substitution by reactive groups affect the resulting physical properties of the crosspolymer. Thus, silicone crosspolymers with particular physical characteristics may be designed merely by varying the proportions or identities of the individual silicone components. In particular embodiments, then, the pharmaceutical composition comprising a corticosteroid further comprises a high molecular weight, low viscosity silicone crosspolymer.

The silicone crosspolymers useful in the pharmaceutical compositions provided herein are those that dry quickly, have a soft, silky feel on the skin and add a luxurious texture to the formulation when initially applied. Any suitable high molecular weight silicone crosspolymer may be employed. The use of crosslinked silicone polymers eliminates the need for a catalyst or crosslinking agent in the corticosteroid formulation. In some embodiments, the preferred molecular weight of the crosspolymer depends upon the desired viscosity of the scar dressing formulation as well as the desired characteristics of quick drying, conformity, texture, and non-tackiness. The silicone crosspolymer can be, for example, a crosspolymer of dimethicone, cyclomethicone, cyclohexasiloxane, or cyclopentasiloxane, or a mixture thereof. Exemplary crosspolymers include Dow Corning® 9040 (cyclomethicone/dimethicone crosspolymer blended with cyclomethicone), Dow Corning® 9506 powder (dimethicone/vinyl dimethicone crosspolymer), or KSG-210 (dimethicone/PEG-10/15 crosspolymer blended with 24% dimethicone) (ShinEtsu Chemical Co. Ltd). Typically, the high molecular weight crosspolymer has a low viscosity of about 50 cSt or less, about 25 cSt or less, or sometimes 5 cSt or less.

In some embodiments, the preferred particle size of the crosspolymer depends upon the desired viscosity of the corticosteroid formulation as well as the desired characteristics of quick drying, scar coverage, conformity, texture, and non-tackiness. In general, the particle size range can be from about 500 nm to about 100 μm. In some embodiments, the particle size ranges from about 1 to about 15 μm. The average particle size can be about 500 nm, about 1 about 3 μm, about 5 μm, about 10 μm, about 15 μm, or greater.

In some embodiments, the composition comprising a silicone crosspolymer further comprises at least one silicone oil. The silicone oil component may be used to keep the crosspolymer from polymerizing or curing before that transition is desired, or may confer desirable texture, volatility, tackiness, or other characteristics to the formulation. The silicone oil may be part of a commercially available crosspolymer product, or may be added to the commercially available crosspolymer. The silicone oils useful in the corticosteroid formulations provided herein have a high nonvolatile content of greater than 70%, greater than 80% or greater than 90%. Suitable silicone oils include super low viscosity silicone fluids such as cyclomethicone, dimethicone, cyclopentasiloxane, cyclohexasiloxane, Botanisil S-19 (PEG-12 dimethicone), or Volasil 7525 (Chemisil Silicones, Inc.; cyclohexasiloxane and cyclopentasiloxane), or a mixture thereof. Thus, the at least one silicone oil is selected from the group consisting of: cyclomethicone, dimethicone, cyclopentasiloxane, cyclohexasiloxane, and PEG-12 dimethicone, and mixtures thereof. The silicone crosspolymer and at least one silicone oil together represent greater than about 70%, about 80%, greater than about 85%, greater than about 90%, or greater than 95% by weight of the corticosteroid formulation.

In some embodiments, the corticosteroid formulation comprising silicone crosspolymers is applied to the desired site while in a substantially flowable state. The formulation as prepared is flowable when administered and thus may be applied to wound surfaces for up to 15 minutes before complete curing. The flowable or substantially flowable state permits the formulation to be custom fit to any contoured or shaped surface. Thus, the formulation is applied to the scar and can be worked with for about 2 minutes to about 15 minutes to cover the scar as necessary. After application, the formulation is smoothed to a desired thickness and becomes substantially tack-free.

The corticosteroid formulation comprising silicone crosspolymers typically forms a membrane having a thickness from about 0.1 mm to about 5 mm upon curing. The membrane can be continuous or substantially continuous over the surface of the scar. The continuous nature of the membrane allows the formulation to act as a bacterial barrier. The formulation is free or at least substantially free of air bubbles. The corticosteroid formulation comprising silicone crosspolymers can be transparent or substantially transparent. Transparency permits visual observation and monitoring of the scar as it continues to heal and improves the cosmetic appearance of the dressing (e.g., renders it less conspicuous). In addition, the silicone crosspolymer formulation adheres to scar tissue and prevents transepidermal water loss from the affected area.

The corticosteroid formulation may also optionally contain one or more therapeutic additives. Such additives include, but are not limited to antimicrobial agents, including antibacterials (such as neomycin, bacitracin, mupirocin, tetracycline, erythromycin, gentamycin, tobramycin, and the like), antivirals (such as acyclovir, pencyclovir, and the like), and antifungals (fluconazole, miconazole, terbinefine, posaconazole, and the like). Suitable scar dressing formulations may contain from about 0.01% to about 20% by weight of at least one therapeutic additive. In a particular embodiment, the therapeutic additive is about 5% or less by weight, about 3% or less by weight, or about 1% or less by weight.

In particular, the corticosteroid formulation may further comprise an anti-inflammatory agent. Suitable anti-inflammatory agents include drug compounds which reduce inflammation or inhibit inflammation-mediated processes. More particularly, preferred anti-inflammatory agents are compounds that inhibit enzymes in the PLA₂ pathway, such as PLA₂ and/or cyclooxygenase-2 (COX₂). Examples of such compounds include diclofenac, meloxicam, ibuprofen, and the like. Further exemplary PLA₂ and/or COX₂ inhibitors include compounds as described in U.S. Pat. Nos. 6,495,596 and 6,998,421. Such compounds include fatty acid-glycerol-PEG compounds such as the glyceryl distearate, glyceryl dioleate, or glyceryl dimyristate derivatives of PEG-12, PEG-23, or PEG-45. In further embodiments, the anti-inflammatory agent is PEG-12 glyceryl distearate, PEG-23 glyceryl distearate, PEG-12 glyceryl dipalmitate, or PEG-12 glyceryl dimyristate. In other embodiments, the anti-inflammatory agent is PEG-23 glyceryl distearate or PEG 12 glyceryl dipalmitate. In still further embodiments, the anti-inflammatory agent is PEG-12 glyceryl dipalmitate.

The term “treat” or “treating” as used herein is intended to refer to administration of a corticosteroid to a subject for the purpose of creating a therapeutic benefit. Treating includes various desirable therapeutic outcomes including reduced healing time, decreased redness or other discoloration, decreased hyperpigmentation, decreased erythma, reduced scar height, reduction or elimination of keloid formation, decreased scar pain, increased patient comfort, improved cosmetic appearance (aesthetic) of the scar, decreased vascularity, increased pliability, or overall improved quality and texture of the healed scar tissue, or any combination of these parameters. The term “subject” refers to a mammalian patient in need of such treatment, such as a cat, dog, horse, cow, or human. In preferred embodiments, the subject is a human patient.

In treatment methods according to the invention, “an effective amount” means an amount or dose sufficient to generally bring about the desired therapeutic benefit in subjects needing such treatment. Effective amounts or doses of corticosteroids may be ascertained by routine methods, such as modeling, dose escalation, or clinical trials, taking into account routine factors, e.g., the mode or route of administration or the particular drug delivery technology used, the pharmacokinetics of the agent, the potency of the corticosteroid, the severity of the wound or scar, the location, age, origin or other characteristics of the wound or scar, the subject's health status, condition, and weight, and the judgment of the treating physician. The total dosage may be given in single or divided dosage units (e.g., BID, TID, QID, or more or less frequently). For topical administration, corticosteroids may be formulated with a pharmaceutical carrier at a concentration of about 0.1% to about 10% by weight of drug. In preferred embodiments, corticosteroid concentration in the formulation is from about 0.05% to about 5% by weight of drug. In further preferred embodiments, the corticosteroid concentration is about 0.1 to about 2% by weight, is about 0.25% by weight, or is about 1% by weight. Suitable individual doses may also be measured by the amount of drug or formulation administered per square centimeter of scar surface area; in such cases, suitable amounts are about 0.1 to 2 grams of drug formulation per square centimeter, or about 0.5 to 1.5 grams of drug formulation per square centimeter. Particularly, the amount of corticosteroid formulation used will be sufficient to cover the entire wound or scar such that the affected area is completely occluded by the formulation.

As discussed above, intralesional corticosteroid injections are usually performed on an open wound at the time the wound is inflicted, e.g., at the end of a surgical procedure. The presently described corticosteroids and formulations are useful at any stage of scar evolution and thus may be applied to new wounds or scars (for example, with treatment beginning immediately following a surgical or dermatological procedure) or old wounds or scars. It has been found that optimal scar healing results for topical corticosteroid formulations are achieved if the wound/scar is treated beginning at a time point after the wound formation, for example two, three, or several days after wound formation, preferably two or three days after wound formation, once the wound is closed, has completed the initial re-epithelization process and begun the collagen rebuilding phase. Thus, the pharmaceutical formulation may be administered to the wound or scar beginning two or three days after wound formation. The formulation is also useful to treat scars during the contraction, maturation or remodeling stages of wound healing. Scars with ages of at least two days, at least a week, or at least 1, 2, 4, 6, 12, 24, 36, or 48 months, and even scars up to 10 years old and beyond may be beneficially treated with corticosteroids and the formulations described herein. The scar can be less than about 1 week old, about 2 weeks old, about 1 month old, about 3 months old, or greater. Scars more than a month old may be referred to herein as “established” scars.

A corticosteroid or formulation comprising a corticosteroid can remain on the scar for any time sufficient to permit healing of and/or resolution of the scar. In particular embodiments of the invention, a corticosteroid or pharmaceutical composition comprising a corticosteroid is administered to the wound or scar once or more than once. If the medication is administered more than once, administration can be once or more than once per day. In further preferred embodiments, the corticosteroid is administered twice daily, or administered three times daily. Preferably, the wound and/or scar is treated for one day, up to one week, up to two weeks, up to four weeks, up to six weeks, up to twelve weeks, or up to 30, 60, 75, 90, 120, or 180 days or longer. In some embodiments, treatment extends for about two to about three months, or about 30, 60, 75, or 90 days, or for longer periods. More particularly, the corticosteroid or pharmaceutical composition is administered two or three times daily; this practice may be repeated for up to 180 days, or for about 90 to about 180 days. In one embodiment, the formulation forms a membrane over the wound that remains in place for at least about 1 day, at least about 2 days, at least about 4 days, at least about 6 days, or at least about 7 days to about 10 days. After the corticosteroid or formulation has been on a scar for a time sufficient to promote and/or substantially complete healing and scar formation, the formulation can removed by gently wiping it from the scar. The treating physician may also alter the frequency of administration as the wound and/or scar heals.

The present invention also contemplates a kit comprising the components of the formulation as disclosed herein and optionally instructions for use.

Exemplary formulations comprise the following ingredients (% by weight):

| | |
|---|---|
| Silicone Crosspolymer | 55-90% |
| Silicone Oil(s) | 5-20% |
| Anti-Inflammatory Agent | 0.03-2% |
| Corticosteroid | 0.05-5% |
| Preservative | 1-4% |

Further exemplary formulations comprise the following ingredients (% by weight):

| | |
|---|---|
| Silicone Crosspolymer | 2-45% |
| Silicone Oil(s) | 5-90% |
| Anti-Inflammatory Agent | 0.2-16% |
| Corticosteroid | 0.1-5% |
| Preservative | 2-50% |
| Ethanol | 2-50% |

Still further exemplary formulations comprise the following ingredients (% by weight):

| | |
|---|---|
| Dimethicone Crosspolymer | 2-45% |
| Cyclomethicone and Cyclopentasiloxane | 5-90% |
| PEG-12 Glyceryl Dimyristate | 0.2-16% |
| Propylene Glycol | 0.1-40% |
| Benzyl Alcohol | 0.1-20% |
| Ethanol | 2-50% |
| Prednisolone Acetate or Methylprednisolone Acetate | 0.1-4% |

The following examples are offered to illustrate but not to limit the invention.

### EXAMPLES

### Example 1

### Wound Healing in Hairless Mice

### A) Wounding Method

Twelve hairless mice (Skh:HR-1) with an average weight of 1.62 kg were treated topically with a local anesthetic (LMX-4, Ferndale Laboratories, Inc.) for dermal anesthesia for 30 minutes prior to wounding. Antiseptics were not used because of the possibility of their local effect on the wound healing process.

Two linear subcutaneous-deep wounds were made bilaterally with a 0.3 mm blade surgical knife on the dorsal side of each subject approximately 10 mm on each side of the spinal column. Because of the loose nature of the hairless mouse epidermis the wounds spread to as wide as 3 mm. Each mouse was housed separately.

### B) Formulations

The test formulation comprised the following ingredients (by weight percent): water (79%), ethanol (18%), prednisolone acetate (1%), and ammonium acryloyldimethyltaurate/VP copolymer (2%).

### C) Treatment

In each test subject, the right wound was assigned to a control (i.e., no treatment) and the left would was treated with a the 1% prednisolone acetate test formulation, applied twice daily (8 hours apart) beginning 1 h after the incision on day 1 and continuing through day 21. The wounds were about 20 mm apart to prevent the treatment gel from migrating from the treatment wound to the control wound.

### D) Healing Evaluation

Healing evaluation was made by measuring wound closure, which was assessed by the width of the wound. Evaluation was made on days 1, 3, 5, 10, and 14. The width of each scar was measured with digital calipers at the widest point while subject was lying motionless on its ventral side. Digital photographs were taken to record healing process. Data for these measurements are presented in Table 1.

TABLE 1
Scar Width (mm) at widest margin

| Subject | Day 1 | | Day 3 | | Day 5 | | Day 10 | | Day 14 | |
|---|---|---|---|---|---|---|---|---|---|---|
| Wound | L | R | L | R | L | R | L | R | L | R |
| 1 | 2.9 | 3.0 | 1.9 | 2.5 | 1.6 | 1.8 | 0.6 | 0.8 | 0.0 | 0.2 |
| 2 | 2.7 | 2.9 | 1.8 | 2.2 | 1.5 | 1.9 | 0.5 | 0.9 | 0.0 | 0.5 |
| 3 | 2.8 | 2.8 | 1.7 | 2.0 | 1.6 | 1.8 | 0.6 | 0.8 | 0.2 | 1.0 |
| 4 | 2.9 | 2.9 | 1.7 | 2.0 | 1.4 | 1.9 | 0.7 | 0.9 | 0.0 | 1.0 |
| 5 | 3.0 | 2.9 | 1.9 | 2.1 | 1.7 | 2.0 | 0.6 | 1.4 | 0.2 | 0.9 |
| 6 | 2.6 | 2.7 | 1.9 | 2.2 | 1.7 | 2.1 | 0.2 | 1.1 | 0.0 | 0.5 |
| 7 | 2.9 | 3.0 | 1.8 | 2.4 | 1.6 | 2.0 | 0.4 | 0.3 | 0.0 | 0.0 |
| 8 | 3.0 | 2.8 | 1.4 | 2.5 | 1.1 | 1.9 | 0.5 | 1.3 | 0.0 | 0.8 |
| 9 | 2.7 | 2.6 | 1.8 | 2.2 | 1.7 | 2.2 | 0.3 | 1.4 | 0.0 | 0.8 |
| 10 | 2.8 | 3.0 | 1.5 | 2.4 | 1.3 | 2.0 | 0.5 | 1.3 | 0.0 | 0.9 |
| 11 | 2.7 | 2.7 | 1.7 | 2.6 | 1.4 | 1.7 | 0.4 | 1.6 | 0.0 | 1.0 |
| 12 | 2.9 | 2.8 | 1.8 | 2.2 | 1.4 | 2.0 | 0.6 | 1.9 | 0.0 | 1.3 |
| Mean | 2.8 | 2.8 | 1.8 | 2.2 | 1.5 | 1.9 | 0.5 | 1.0 | 0.0 | 0.7 |

(L = treatment group; R = control group)

Healing in the treatment subgroup was faster than the control group as indicated by differences in wound-width over a 14-day period. By day 14, mean wound width was 0 mm for the treatment group and 0.7 mm for the control group. Generally, throughout the healing time treated scars showed greater closure at the measured time points compared to the control scars. The mean differences in wound width were: day 3, 0.4 mm; day 5, 0.4 mm; day 7, 0.5 mm; day 10, 0.5 mm; and day 14, 0.7 mm.

### E) Scar Evaluation

Each mouse was kept in its cage during days 14-21. On day 21, each scar was assessed according to the Vancouver Scale (18 points) under magnification by two observers independently and the scores were averaged and recorded. Progressive re-epithelialization of the wound surface from the wound edges continued until the wound surface was completely closed at a time between days 14 and 21. Data for these assessments are presented in Table 2.

TABLE 2
Averaged Vancouver Scale Scar Scoring at Day 21

| Subject | Vascularity | | Pliability | | Pigmentation | | Height | |
|---|---|---|---|---|---|---|---|---|
| Wound | L | R | L | R | L | R | L | R |
| 1 | 0 | 0 | 1.5 | 2 | 1 | 2 | 0 | 0 |
| 2 | 0 | 1 | 1 | 5 | 1 | 2 | 0 | 0 |
| 3 | 0 | 1 | 1 | 5 | 1 | 1 | 0 | 0 |
| 4 | 0 | 2 | 1 | 3 | 1 | 1 | 0 | 0 |
| 5 | 1 | 1 | 1 | 2 | 1.5 | 1.5 | 0 | 0 |
| 6 | 0 | 2 | 1 | 5 | 1 | 2 | 0 | 1 |
| 7 | 0 | 1 | 1 | 5 | 1 | 1.5 | 0 | 0 |
| 8 | 0 | 1 | 1 | 5 | 1 | 2 | 0 | 1 |
| 9 | 0 | 0 | 1 | 5 | 1 | 1 | 0 | 0 |
| 10 | 1 | 2 | 1.5 | 2 | 1 | 1 | 0 | 1 |
| 11 | 0 | 1 | 1 | 5 | 1 | 1 | 0 | 0 |
| 12 | 0 | 1 | 1 | 5 | 1 | 2.5 | 0 | 0.25 |
| Mean | 0.16 | 1.08 | 1.1 | 4.1 | 1.1 | 1.5 | 0 | 0.41 |

(L = treatment group; R = control group)

### F) Results

The wounds appeared to heal progressively during the evaluation and healing period. No apparent retarding of any phase of healing was observed. Grossly, wound healing looked different between the two subgroups. The wounds on the right side of the spinal column (control wounds) had a classic wound crusting, a fibrous cacophony consisting mostly of fibrin, macrophages and neutrophils. The degree of crusting varied within the subgroup but each wound on the control side exhibited some of this crusting phenomenon during the first 14 days. The treated wounds on the left dorsal side of each subject were clear of crusting, infiltrates, and exudates and the wounds appeared fresher yet closure also appeared more rapid. Neither the control nor the treated wounds showed signs of infection, although more infiltration was observed for the control wounds over the treated wounds.

Another distinctive difference between the control and treated wounds was that 8 out of the 12 treated scars healed with some degree of contracture. Significantly, the quality of the healed epidermal layer treated showed less contracture. This feature began early in the process, becoming evident by day 5. The treated wounds displayed less redness and a finer marginal line between the two sides of the initial wound.

In our global assessment of the scars on day 21 using the Vancouver Scale there was a significant difference in pliability, pigmentation and vascularity between the two subgroups. Vascularity returned to virtually normal in the treated areas and was almost normal in the control areas. Pigmentation was marginally better in the treatment group but both groups had some degree of hypopigmentation. The height of the wound returned to almost the same height as the normal epidermal layer; the height of the scars in both groups was very minimal and in most cases too small to measure.

### Example 2

### Wound Healing in Hairless Mice

### A) Wounding Method

Twelve hairless mice (Skh:HR-1) with an average weight of 1.62 kg were treated topically with a local anesthetic (LMX-4, Ferndale Laboratories, Inc.) for 30 minutes prior to wounding for dermal anesthesia. Antiseptics were not used because of the possibility of their local effect on the wound healing process.

The mice were subdivided into two groups, A and B. Each mouse received three linear, 10 mm long, subcutaneous-deep incision wounds parallel to the spinal column. The first incision was near the spinal column on the left side (C), one at a position 10 mm away from the first on the same side (L), and one on the right side about 10 mm from the center line or spinal column (R). All incision wounds were made with a 0.3 mm blade surgical blade. Because of the loose nature of the hairless mouse epidermis the wounds spread to as wide as 3 mm. Each mouse was housed separately.

### B) Formulations

The following formulations were used in this example (% by weight):

1) 0.25% Formulations: water (79.75%), ethanol (18%), methylprednisolone acetate or prednisolone acetate (0.25%), and ammonium acryloyldimethyltaurate/VP copolymer (2%).

2) 1% Formulations: water (79%), ethanol (18%), methylpredinisolone acetate or prednisolone acetate (1%), and ammonium acryloyldimethyltaurate/VP copolymer (2%).

### C) Treatment

In Group A, wounds C and L were treated with the 0.25% and 1% methylprednisolone acetate formulations, respectively, and wound R was the control or untreated wound. In Group B, wounds C and L were treated with the 0.25% and 1% prednisolone acetate formulations, respectively and wound R was the control. The study drug (methylprednisolone or prednisolone) was applied to the treatment wounds twice daily (8 hours apart) beginning 1 h after the incision on day 1 and continuing through day 21. The wounds were far enough apart to avoid the spread of the treatment gel from the treated wound to the control wound. No additional physical barrier, such as a gauze bandage, was used.

### D) Healing Evaluation

Healing evaluation was made by measuring wound closure, which was assessed by the width of the wound. The width of each scar was measured with digital calipers at the widest point while subject was lying motionless, on its ventral side. Digital photographs were taken to record the healing process. The mean width of wound was recorded on days 1, 3, 5, 7, 9, 11, 13, 15, 17, 19 and 21. Data from these measurements are presented in Tables 3 and 4.

TABLE 3
Group A (n = 12) Mean Wound Width at Widest Margin (mm)

| Day/Wound | L | C | R (Control) |
|---|---|---|---|
| 1 | 3.0 ± 0.2 | 3.1 ± 0.3 | 3.0 ± 0.2 |
| 3 | 2.4 ± 0.3 | 2.5 ± 0.2 | 2.7 ± 0.4 |
| 5 | 1.9 ± 0.4 | 2.0 ± 0.3 | 2.4 ± 0.6 |
| 7 | 1.4 ± 0.2 | 1.5 ± 0.2 | 2.1 ± 0.5 |
| 9 | 0.9 ± 0.3 | 1.0 ± 0.3 | 1.8 ± 0.4 |
| 11 | 0.5 ± 0.1 | 0.5 ± 0.2 | 1.5 ± 0.5 |
| 13 | 0.1 ± 0.2 | 0.1 ± 0.3 | 1.2 ± 0.6 |
| 15 | 0 | 0 | 0.9 ± 0.4 |
| 17 | 0 | 0 | 0.3 ± 0.3 |
| 19 | 0 | 0 | 0 |
| 21 | 0 | 0 | 0 |

TABLE 4
Group B (n = 12) Mean Wound Width at Widest Margin (mm)

| Day/Wound | L | C | R (Control) |
|---|---|---|---|
| 1 | 3.0 ± 0.2 | 3.1 ± 0.3 | 3.0 ± 0.4 |
| 3 | 2.4 ± 0.3 | 2.5 ± 0.2 | 2.7 ± 0.3 |
| 5 | 1.8 ± 0.4 | 1.7 ± 0.2 | 2.3 ± 0.5 |
| 7 | 1.5 ± 0.2 | 1.4 ± 0.2 | 2.0 ± 0.3 |
| 9 | 1.0 ± 0.2 | 1.1 ± 0.2 | 1.7 ± 0.5 |
| 11 | 0.6 ± 0.2 | 0.6 ± 0.2 | 1.4 ± 0.4 |
| 13 | 0.2 ± 0.3 | 0.1 ± 0.2 | 1.1 ± 0.5 |
| 15 | 0 | 0 | 0.9 ± 0.4 |
| 17 | 0 | 0 | 0.4 ± 0.5 |
| 19 | 0 | 0 | 0.1 ± 0.2 |
| 21 | 0 | 0 | 0 |

### E) Scar Evaluation

Each mouse was kept in its cage during days 14-21. On day 21, each scar was assessed according to the Vancouver Scale (18 points) under magnification by two observers independently and the scores were averaged and recorded. Progressive re-epithelializaton of the wound surface from the wound edges continued until the wound surface was completely closed at a time between days 14 and 21. Data for these assessments are presented in Tables 5 and 6. Gross evaluation of the scars for wound closure and presence of infection at day 60 and day 90 revealed better scar healing results for test subjects compared to control subjects.

TABLE 5
Group A-Mean Vancouver Scale Scar Scoring at Day 21

| WOUND | L | C | R (Control) |
|---|---|---|---|
| Vascularity | 0 | 0 | 1 |
| Pliability | 1.5 | 2.0 | 5 |
| Pigmentation | 1 | 1 | 2 |
| Height | 0 | 0 | 0 |

TABLE 6
Group B-Mean Vancouver Scale Scar Scoring at day 21

| WOUND | L | C | R (Control) |
|---|---|---|---|
| Vascularity | 0 | 0 | 1 |
| Pliability | 1.0 | 2.5 | 5 |
| Pigmentation | 1 | 1 | 2.5 |
| Height | 0 | 0 | 0 |

The same qualitative observations were made between the treatment and control groups as described above for Example 1.

### Example 3

### Exemplary Corticosteroid Formulations

The following are exemplary formulations for the invention.

Formulation 1:

| Ingredient | Concentration (w/w %) |
|---|---|
| Purified water (USP) | 63.978 |
| Ethanol (USP) | 30.0 |
| PEG-12 Glyceryl dimyristate or dipalmitate | 3.00 |
| Ethoxydiglycol | 1.00 |
| Butylene glycol | 1.00 |
| Methylprednisolone acetate (USP) | 1.00 |
| Carbopol ETD 2001 | 0.012 |
| Citric acid | 0.01 |

Formulation 2:

| Ingredient | Concentration (w/w %) |
|---|---|
| Purified water (USP) | 82.8 |
| Isopropyl palmitate | 1.00 |
| Caprilic/capric triglyceride | 1.00 |
| Sorbitan stearate | 1.00 |
| Methylprednisolone acetate (USP) | 0.25 |
| Corn oil | 0.5 |
| Stearic acid | 1.5 |
| Cetyl calohol | 3.5 |
| Glyceryl stearate | 2.5 |
| PEG-100 stearate | 1.5 |
| Methylparaben | 0.25 |
| Propylparaben | 0.1 |
| Oleic acid | 0.1 |
| Phenoxyethanol | 1.00 |
| Carbopol Ultrez 10 | 3.00 |

Formulation 3:

| Ingredient | Concentration (w/w %) |
|---|---|
| Purified water (USP) | 70.50 |
| Glycerine | 22.00 |
| PEG-12 glyceryl distearate | 2.50 |
| Carbopol ETD 2020 | 0.50 |
| Propylene glycol | 1.00 |
| Prednisolone acetate (USP) | 1.00 |
| Cholesterol | 0.25 |
| Trolamine | 0.25 |
| Benzyl alcohol | 2.00 |

Formulation 4:

| Ingredient | Concentration (w/w %) |
|---|---|
| Cyclomethicone | 5-25% |
| Cyclopentasiloxane | 10-90% |
| Dimethicone Crosspolymer | 2-45% |
| PEG-12 Glyceryl Dimyristate | 0.2-16% |
| Propylene Glycol | 0.1-40% |
| Benzyl Alcohol | 0.1-20% |
| Ethanol | 2-50% |
| Methylprednisolone Acetate | 0.1-4% |

Formulation 5:

| Ingredient | Concentration (w/w %) |
|---|---|
| KSG-210 | 95.7500% |
| Botanisil S-19 | 0.5000% |
| PEG-12 glyceryl dimyristate | 0.5000% |
| Benzyl Alcohol | 0.7500% |
| Purified Water | 1.5000% |
| Methylprednisolone Acetate | 1% |

Formulation 6:

| Ingredient | Concentration (w/w %) |
|---|---|
| Dow Corning ® 9040 | 83.2500% |
| Cyclomethicone | 15.0000% |
| PEG-12 glyceryl dimyristate | 0.5000% |
| Glycerin | 1.0000% |
| Prednisolone Acetate | 0.25% |

Formulation 7:

| Ingredient | Concentration (w/w %) |
|---|---|
| Dow Corning ® 9506 Powder | 13.0000% |
| Volasil 7525 | 85.2500% |
| PEG-12 glyceryl dimyristate | 0.5000% |
| Glycerin | 1.0000% |
| Methylprednisolone Acetate | 0.25% |

### Example 4

### Wound/Scar Treatment Following Surgery

Following abdominoplasty or breast reconstruction surgery, a patient applies 0.25% or 1% topical methylprednisolone acetate cream to the wound/scar twice daily until the wound heals and/or for up to 12 weeks. For each application, the patient first cleans the affected area with a suitable skin cleanser. The medicated cream is applied by rubbing a thin layer on and around the affected area.

Wound/scar healing is measured using the Vancouver Scar Scale for pigmentation, height, vascularity, and pliability. Erythema is measured using a Konica-Minolta Dermatospectrometer, which uses the principles of color measurements as established by the Commission International d'Eclairage (CIE), using the terms specified by the CIE in 1976 of L*a*b (CIELAB) for color space parameters. See Niessen, F. B. et al. “The use of silicone occlusive sheeting (Sil-K) and silicone occlusive gel (Epiderm) in the prevention of scar formation.” Plast. Reconstr. Surg. 1998, 102(6), 1962-72. The L* parameter: represents the reflection and ranges from 0, theoretical black, to 100 for perfect white. A* represents the red-green spectrum (low values represent less red in a scar). B* represents the yellow-blue spectrum. Healing is also assessed using a Durometer/pneumatonometer BTC 2000: to measure pliability. Measurements are taken by a physician after each month of treatment.

### Example 5

### Clinical Evaluation of Corticosteroid Formulation Using Silicone Crosspolymers

Formulation 4 (“test formulation”) was used to assess scar healing properties as compared to a control formulation comprising silicone oil (e.g., cyclomethicone, but lacking silicone crosspolymers) and hydrocortisone (“control formulation”), in a prospective, randomized, controlled Phase III clinical trial. Patients applied the test formulation to one randomized side of a scar and the control formulation to the other half of the affected area. Both formulations were applied twice daily for 60 days. Digital photography and both patient and physician evaluation was completed at 0, 30, and 60 days after treatment was initiated. Scars were rated by physicians according to a modified Vancouver Scar Scale which evaluates multiple characteristics of a scar included degree of pigmentation, pliability, scar height, presence of vascularity, and patient assessment of pain and pruritus.

Nineteen of 21 patients recruited and enrolled in the study completed the 8-week course of treatment. Seventeen females and two males completed the study with a mean age of 41.5 years (range 22-71). All patients participating in the study were Fitzpatrick skin types II-IV (Type II: 14 patients, Type III: 6 patients, Type IV: 1 patient). All patients were nonsmokers.

The mean age of the scar treated was 17 months (range 1-98 months). Scars treated included those resulting from: abdominoplasties (3), Cesarean section (1), breast reduction (8), abdominal laparotomy (1), tattoo scar (1, dropped out), brachioplasty (2), traumatic injury (elbow, 1), ACL repair (knee, 1), rhytidectomy (face lift, postauricular, 2), and Mohs surgery (lower leg, 1).

Analysis of variance (ANOVA) was completed for statistical analysis of the test and control formulations, comparing the modified Vancouver Scar Scale (VSS) scores of these two groups at baseline (day 0) to the final visit (day 60). The improvement in the VSS for the test formulation group was found to be statistically significant, with a baseline VSS score of with a change in VSS of 6.43 and a day 60 score of 4.87 (p-value 0.046). The improvement in the control group was not found to be statistically significant (baseline score 6.0, day 60 score 4.63, p-value 0.053). There was no statistically significant improvement by a paired t-test between the test and control groups.

The patient feedback on both products was strikingly different with similar comments elicited from numerous patients. At the two-month follow-up visit, patients were asked an open-ended question about whether they had any comments about their experience with either formulation. Ten of the 12 patients who provided feedback reported they preferred the test formulation to the control. In all ten cases, the patients indicated they preferred the feel and/or smell of the test formulation over the control. The majority of these patients stated that the test formulation was easy to apply and blended well into the skin while the control formulation peeled off the skin, sometimes leaving material on their clothing or undergarments.

This example demonstrates the corticosteroid/silicone crosspolymer formulation led to a statistically significant improvement in scar appearance over a two-month period according to a modified Vancouver Scar Scale when compared to a formulation lacking the crosspolymers and anti-inflammatory agent. Patients much preferred the feel, smell, and ease of application of the subject formulation.

While the invention has been explained in relation to its preferred embodiments, it is to be understood that various modifications thereof will become apparent to those skilled in the art upon reading the specification. Therefore, it is to be understood that the invention disclosed herein is intended to cover such modifications as fall within the scope of the appended claims.

1. A method of treating a subject with a wound or a scar comprising topically administering to the wound or scar an effective amount of a pharmaceutical composition comprising a corticosteroid.

2. The method of claim 1, wherein the corticosteroid is prednisolone acetate or methylprednisolone acetate.

3. The method of claim 1, wherein the pharmaceutical composition is administered beginning two or three days after wound formation.

4. The method of claim 3, wherein the pharmaceutical composition is administered two or three times daily.

5. The method of claim 4, wherein the pharmaceutical composition is administered for up to 180 days.

6. The method of claim 4, wherein the pharmaceutical composition is administered for about 90 to about 180 days.

7. The method of claim 1, wherein the pharmaceutical composition further comprises a high molecular weight, low viscosity silicone crosspolymer.

8. The method of claim 1, wherein the pharmaceutical composition further comprises an effective amount of an anti-inflammatory agent.

9. The method of claim 7, wherein the pharmaceutical composition further comprises an effective amount of an anti-inflammatory agent.

10. The method of claim 8, wherein the anti-inflammatory agent is a phospholipase A₂ and/or cyclooxygenase-2 inhibitor.

11. A pharmaceutical composition comprising a corticosteroid and a high molecular weight, low viscosity silicone crosspolymer.

12. The pharmaceutical composition of claim 11, wherein the corticosteroid is methylprednisolone acetate or prednisolone acetate.

13. The pharmaceutical composition of claim 11, wherein the crosspolymer is a crosspolymer of dimethicone, cyclomethicone, cyclohexasiloxane, or cyclopentasiloxane, or a mixture thereof.

14. The pharmaceutical composition of claim 11, further comprising at least one silicone oil.

15. The pharmaceutical composition of claim 14, wherein the at least one silicone oil is selected from the group consisting of: cyclomethicone, dimethicone, cyclopentasiloxane, cyclohexasiloxane, and PEG-12 dimethicone, and mixtures thereof.

16. The pharmaceutical composition of claim 11, further comprising an anti-inflammatory agent.

17. The pharmaceutical composition of claim 16, wherein the anti-inflammatory agent is a phospholipase A₂ and/or cyclooxygenase-2 inhibitor.
